(19) United States
(12) Patent Application Publication
(10) Pub. No.: US 2008/0196717 A1
(43) Pub. Date: Aug. 21, 2008

(54) Powder Inhaler

(75) Inventors: Leon Gradon, Warsaw (PL); Arkadiusz Moskal, Warsaw (PL); Tomasz Sosnowski, Warsaw (PL)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXOSMITHKLINE PHARMACEUTICALS S.A., Poznan (PL)

(21) Appl. No.: 11/575,876

(22) PCT Filed: Sep. 8, 2005
(86) PCT No.: PCT/PL05/00059

§ 371 Date: Mar. 10, 2008

(30) Foreign Application Priority Data
Sep. 23, 2004 (PL) P.370285

(51) Int. Cl. A61M 15/00; A61M 16/10

(52) U.S. Cl. 128/203.15

(57) ABSTRACT

The invention consists of a powder inhalator for administrating medications. The inhalator, according to the invention, has a chamber with a mechanism for opening the medication capsule and an aerodynamic chamber in which the medication particles are separated from the currier capsules. In the aerodynamic chamber. On the opposite side to the mouthpiece opening, there are air inlet ducts which form a multidirectional system of air ducts. The air ducts are shaped in a way which makes the air that flows through them create at least three streams. Due to the intersecting of the air streams the air flow is strongly turbulent even if low speeds are involved. Consequently the separation of the active substance particles form the carrier surface is very effective.

The invention consists of a powder inhalator for administrating medications. Powder inhalators are used for administering specified amounts of pharmaceutically active substances or their mixtures in the form of subtly broken up particles into the human bronchial tree.

Single and multi-dose powder inhalators are commonly used. There differ in terms of construction and operation. In known powder inhalators the active substance particles are sucked in by users while they inhale.

The therapeutic effect of a medication depends on the amount of and the place where the active substance particles are deposited in the human bronchial tree. If the active particles are deposited in the oral cavity, the throat or in the upper sections of the bronchial tree the therapeutic effect is insignificant. If, however, the active substance particles reach the lower parts of the bronchial tree the therapeutic effectiveness instantly grows. The smaller the active substance particle and the more intensive the air flow are the deeper the active substance particles are deposited. The best effects are achieved when the size of the active substance particle does not exceed 5 μm. Such small particles, however, quickly agglomerate and cannot be stored for a longer time. In order to provide appropriate duration of the subtle breaking up of the active substance its particles are set onto larger particles of pharmaceutically neutral substances, e.g. lactose particles. The connection between the small active substance particles and the lactose particle is stable during storage and therefore the particles of the medication do not agglomerate. The connection is, however, weak enough to quickly disintegrate due to the movement of air. While inhaling the subtle active substance particles are separated from the carrier particle. Being larger and heavier the carrier particles settle in the oral cavity, whereas the active substance particles are lifted by the stream of air and deeply penetrate the bronchial tree. Consequently the active substance is deposited in the deeper sections of the bronchial tree.

While using each inhalator inside it is accumulated the remains of the active substance and the carrier. This has an effect on the user since after a longer period of usage the inhalator may contain active substance catabolites, oxidation products etc. Therefore, it is recommended to periodically clean the inhalator.

In U.S. Pat. No. 3,991,761 and EP0005585 patent descriptions powder inhalators have been revealed with a chamber in which a capsule containing medication particles is inserted as well as an extended duct which is also used as a mouthpiece. The chamber with the capsule is separated from the air duct with a perforated partition. Before the process of inhaling begins the capsule with the medication is opened by piercing with needles which are located at the two semi-rounded ends of the capsule. The medication is released from the capsule as a result of its rotation caused by the flow of inhaled air. The particles of the medication which are set on the carrier particles are swept away by the air flow the speed of which depends on the force of inhaling. The effectiveness of separating the active substance particles from the carrier depends on the speed and type of air stream, particularly on whether the flow is laminar or turbulent. The perforated partition between the capsule chamber and the duct fulfils the role of an element which enforces a turbulent flow and this in turn makes the separation of the active substance particles from the carrier easier. The disadvantage of these solutions is the incomplete emptying of capsules of the medication particles and, thus, the lack of repeated doses of the medication taken by the user. Additionally, the high resistance of air flow through the perforated partition make proper inhalation more difficult, particularly for elderly people whose breathing is not so efficient as well as for children. Another disadvantage is the perforated partition which is an obstacle that limits the amount of the medication inhaled with the air stream. These inhalators are featured by a relatively high flow resistance which limits its usage by children and people with significant impairments of the respiratory system.

In the British GB 2165159 patent description a powder inhalator was revealed in which a specified volume of medication is administered from a dispenser into the inhalation chamber by means of a specially shaped dial. The inhalation chamber is equipped with an air inflow duct and an air duct which transmits the medication particles to the respiratory system of the user. In this duct there is a narrowing which changes the character of the air flow from laminar to turbulent. The disadvantage of this solution is the unequal dosing of the medication since, depending on the sedimentation level of the powder, its apparent powder density changes and the successive doses which are measured in terms of volume vary in their contents of the medication. Changing the character of the flow from laminar to turbulent in the narrowing depends on the speed of the air flow and, consequently, in relation to people with significant impairments of the respiratory system as well as in relation to children the air flow is only slightly turbulent and this considerably reduces the effectiveness of separating the subtle medication particles form the carrier particles.
In PCT WO03/103563 patent description a powder inhalator was revealed containing a duct into which air is let in through numerous inlets which are of various shapes and positioning. A powdered preparation for inhalation is placed in the duct and while the user inhales, the air which enters the duct through the inlets sweeps away the medication particles which are then further inhaled by the patient. The particular inlets may be covered with fingers while the user inhales and this controls the air flow. The disadvantage of this solution is the chance character of the air flow and, consequently, unrepeated air flow during successive inhalations and varied doses and places of depositing the medication.

In PL 171269 patent description a separator was revealed for separating the active substance particles form the carrier. The disadvantage of this solution is its complicated structure which makes it difficult to clean the separator.

The air of the invention is developing an inhalator with a simple construction which enables the proper process of inhalation and the proper depositing of medications in the bronchial tree, particularly in relation to patients with significant impairments of the respiratory system.
The inhalator, according to the invention, has a chamber with a mechanism for opening the medication capsule in which the process of opening and emptying the medication capsule takes place as well as an aerodynamic chamber in which the medication particles are separated from the carrier capsules. The aerodynamic chamber may also be used as a mouthpiece. In the chamber with the mechanism for opening the medication capsule there is a capsule seat in which the medication capsule is placed as well as a piercing point and a reverse spring. The seat of the capsule is located above the inlet in the aerodynamic chamber. After the capsule is pierced the powder with the medication is spilled directly into the aerodynamic chamber where it rests on its lower surface.
Between the chamber with the mechanism for opening the capsule and the aerodynamic chamber there may be a perforated partition which makes it impossible for slivers of the capsule to get inside the latter chamber.
The aerodynamic chamber is constituted by a hollowing in the stem of the inhalator. This may be of any shape. The hollowing makes it impossible for the powder to move when the position of the inhalator is changed. It is preferable if the hollowing has rounded sides. In the lower part of the aerodynamic chamber, under the inlet which connects it to the powder dispenser, there is a specially shaped seat on which the powder rests.
The aerodynamic chamber may also be used as a moughpiece.

In the aerodynamic chamber, on the opposite side to the mouthpiece opening, there are air inlet ducts which form a multidirectional system of air ducts. The air ducts are shaped in a way which makes the air that flows through them create at least three streams, out of which at least one flows in from the opposite side of the aerodynamic chamber outlet and at least two flow in symmetrically from two sides forming an acute angle with the longitudinal axis of the aerodynamic chamber.

An advantageous effect is achieved when the air stream directed from the opposite side of the air inlet and the longitudinal axis of the aerodynamic chamber form an acute angle which is not narrower than 5° and not wider than 75° as well as when the side streams and the longtudinal axis of the aerodynamic chamber form two equal angles which are not narrower than 5° and not wider than 75°.
The amount of air flowing in the particular streams has a significant effect on the inhalation process. Therefore, it is advantageous when the ratio of the surface area of sections of the ducts which transmit the air to the stream that is directed from the opposite side of the air outlet and the total surface areas of sections of the ducts which transmit the air to the side streams is between 0.5 and 2.
The ducts which transmit the air are shaped in such a way that the axes of the air streams which flow into the aerodynamic chamber intersect at a point which is not further than the place where the medication powder is contained. The best effect is achieved when the axes of these streams intersect just before the place in the aerodynamic chamber where the medication powder is contained.
The intersecting axes of the air streams lift the particles of the medication which are broken up into carrier particles and active substance particles by the intensive turbulent movement.
Due to the intersecting of the air streams the air flow is strongly turbulent even if low speeds are involved. Consequently the separation of the active substance particles form the carrier surface is very effective.

The object of the invention is pictured on one possible (but not the only one) production example in the diagram in which

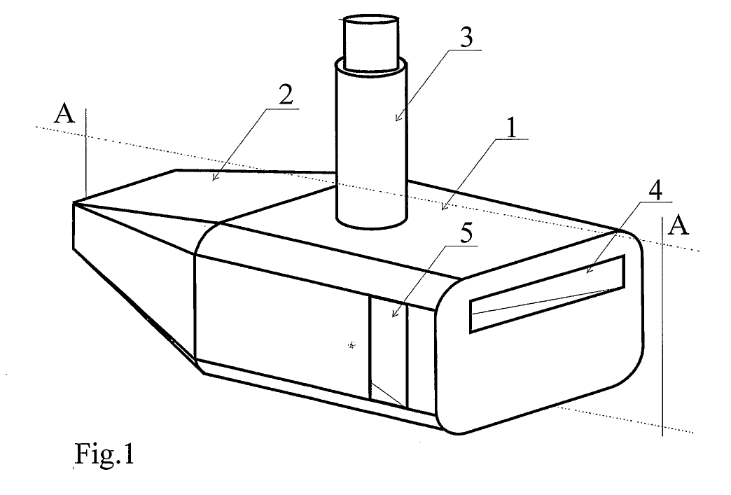

FIG. 1 presents a general view of the inhalator,

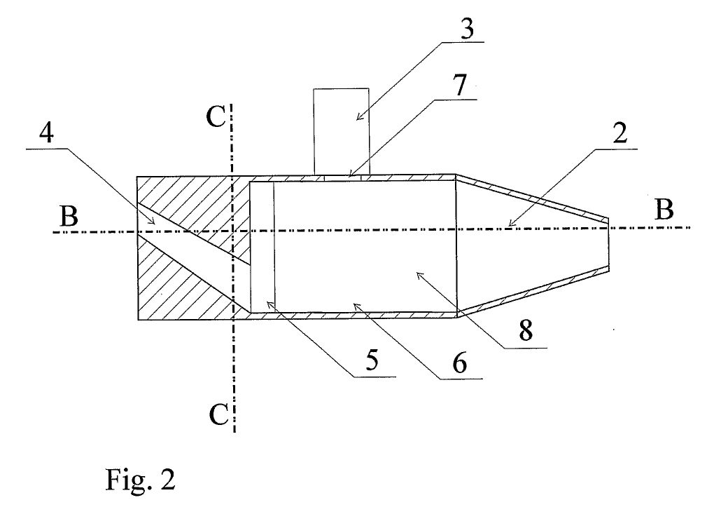

FIG. 2 presents an A-A plane section,

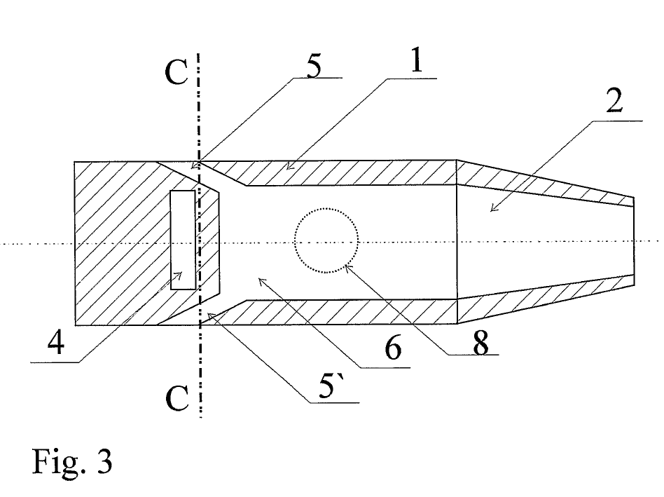

FIG. 3 presents a B-B plane section and

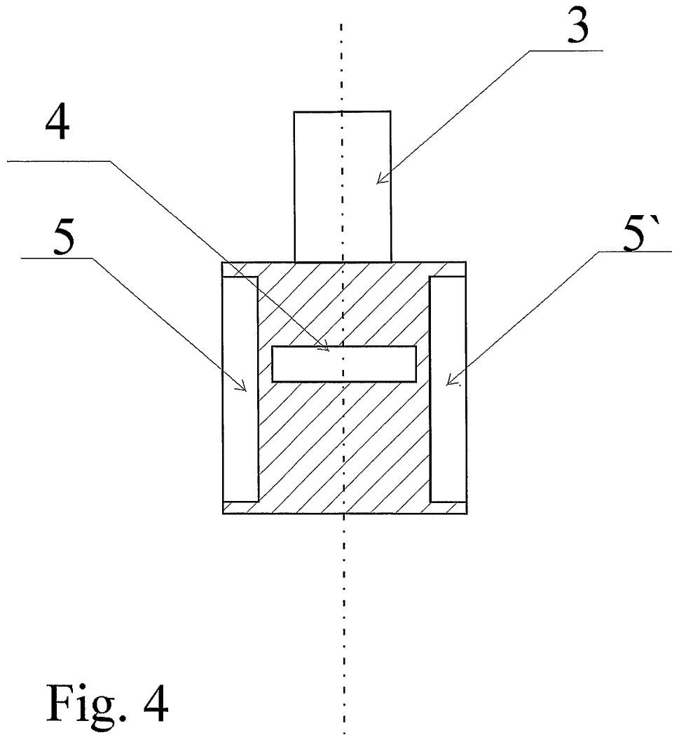

FIG. 4 presents a C-C section.

The inhalator consists of a stem 1 with a mouthpiece 2 and a chamber for opening and emptying capsules 3. In the stem there are ducts 4, 5, 5′ which transmit air into the aerodynamic chamber 6 and an opening 7 which connects the chamber for openingand emptying capsules 3 with the aerodynamic chamber 6.

In the chamber for opening and emptying capsules 3 there is a capsule which is being opened containing powdered medication which falls through the opening 7 to the aerodynamic chamber 6 and rests on its lower surface at a point 8 that is directly under the opening 7.

While inhaling through ducts 4, 5, 5′ air is sucked in. Duct 4 which transmits air to the chamber from the opposite direction of the mouthpiece 2 outlet is shaped in such a way that the axis of air which leaves this duct forms an acute angle with the lower surface of the aerodynamic chamber. The side ducts 5, 5′ are located symmetrically on both sides of the main duct and the axes of air streams which leave them form an acute angle with the axis of the main air stream. The axes of all the air streams intersect in the aerodynamic chamber 6 before the place where the powdered medication is contained 8. A perforated partition may be placed in the opening 7 which connects the chamber for opening and emptying capsules 3 and the aerodynamic chamber 6. This protects the aerodynamic chamber 6 from being entered by capsule particles which may be torn from the capsules while they are being opened.

What is claimed is:

1. An inhalator for inhaling powdered medications with an aerodynamic chamber in which the inhalation powder is placed, wherein there are ducts which transmit the drawn in air streams into the aerodynamic chamber from at least three directions from the side opposite to the outlet of the aerodynamic chamber which ends in a mouthpiece, and the air streams are crossing in the aerodynamic chamber.

2. An inhalator according to claim 1, wherein the air stream that flows into the aerodynamic chamber from the axially opposite direction to the mouthpiece outlet is the main air stream.

3. An inhalator according to claim 2, wherein on both sides of the main air stream there are side air streams.

4. An inhalator according to claim 3, wherein the side air streams and the axis of the main air stream form an acute angle.

5. An inhalator according to claim 4, wherein the axis of the main air stream lies in a surface which is diagonal to the surface on which the inhalation powder is placed.

6. An inhalator according to claim 2, wherein the main air stream and the surface on which the inhalation powder is placed form an angle between 5 and 75°.

7. An inhalator according to claim 6, wherein the side air streams are transmitted to the aerodynamic chamber from the sides and form an angle between 5 and 75° with the axis of the main air stream.

8. An inhalator according to claim 7, wherein the air streams intersect at a point which is not further than the place where the medication powder is contained.

9. An inhalator according to claim 7, wherein the air streams intersect at point where the medication powder is contained.

10. An inhalator according to claim 9, wherein the air streams intersect before the place where the medication powder is contained.

11. An inhalator according to claim 10, wherein the air is transmitted through ducts and wherein the ratio of the total cross sections of the ducts, which transmit the main air stream to the total cross sections of the ducts which transmit the air into the side streams is between 0.5 and 2.

12. An inhalator according to claim 11, wherein the total cross sections of all the ducts which transmit air into the aerodynamic chamber are lower than or equal to the cross section of the surface of the aerodynamic chamber outlet.
